(19) United States
(12) Patent Application Publication
(10) Pub. No.: US 2024/0315480 A1
(43) Pub. Date: Sep. 26, 2024

(54) A COFFEE BEAN GRINDING APPARATUS

(71) Applicant: BREVILLE PTY LIMITED, Alexandria (AU)

(72) Inventors: Lochana Subasekara WIDANAGAMAGE DON, Alexandria (AU); Con PSAROLOGOS, Alexandria (AU)

(21) Appl. No.: 18/573,166

(22) PCT Filed: Jun. 24, 2022
(86) PCT No.: PCT/AU2022/050643

§ 371 Date: Dec. 21, 2023

(30) Foreign Application Priority Data
Jun. 25, 2021 (AU) 2021901928

(51) Int. Cl. A47J 31/42; A47J 42/44; A47J 42/50

(52) U.S. Cl. CPC A47J 31/42; A47J 42/44; A47J 42/50

(57) ABSTRACT

There is disclosed herein a coffee bean grinding apparatus including: a transmitter for emitting a signal; a receiver for receiving at least a portion of the signal; a hopper for containing coffee beans, wherein at least a portion of the coffee beans contained by the hopper is located between the transmitter and receiver; a grinding mechanism for grinding coffee beans fed by the hopper; and a controller, in electrical communication with the transmitter, the receiver and the grinding mechanism, configured to: receive, from the receiver, a measurement indicative of a proportion of the signal received from the transmitter; determine, based on the measurement, whether the hopper contains at least a threshold amount of coffee beans; and in response to determining that the hopper contains less than the threshold amount of coffee beans, perform one or more detection functions.

### TECHNICAL FIELD

The present invention relates to a coffee bean grinding apparatus.

### BACKGROUND

Coffee bean grinders generally include a hopper which contain coffee beans fed to a grinding mechanism. Over time, a level of the coffee beans contained in the hopper reduces such that the hopper needs to be refilled with more coffee beans. A known coffee grinder uses a single detection device including a light emitting diode (LED) and a photodetector. A light that is emitted by the LED can be at least partially reflected by an internal surface of the hopper when an insufficient amount of coffee beans are contained in the hopper, and the reflected proportion of light can be measured by the photodetector. However, this faces a number of issues. Firstly, the magnitude of the light when reflected makes accurate measurements difficult. Further, over time, the internal surface of the hopper can become covered in coffee bean powder which inhibits the reflection of the emitted light, resulting in the device incorrectly detecting that the hopper contains a sufficient amount of coffee beans.

### SUMMARY

It is an object of the present invention to substantially overcome or at least ameliorate one or more disadvantages of existing arrangements or provide a useful alternative.

In a first aspect, there is provided a coffee bean grinding apparatus including: a transmitter for emitting a signal; a receiver for receiving at least a portion of the signal; a hopper for containing coffee beans, wherein at least a portion of the coffee beans contained by the hopper is located between the transmitter and receiver; a grinding mechanism for grinding the coffee beans fed by the hopper; and a controller, in communication with the transmitter, the receiver and the grinding mechanism, configured to: receive, from the receiver, a measurement indicative of a proportion of the signal received by the receiver; determine, based on the measurement, whether the hopper contains at least a threshold amount of coffee beans; and in response to determining that the hopper contains less than the threshold amount of coffee beans, perform one or more detection functions.

In certain embodiments, the transmitter is located within the hopper.

In certain embodiments, the receiver is located: within the hopper; or within the grinding mechanism coupled to the hopper.

In certain embodiments, the coffee bean grinding apparatus includes a user feedback device, wherein the one or more detection functions include the controller controlling the user feedback device to provide an alert indicative of determining that the hopper contains less than the threshold amount of coffee beans.

In certain embodiments, the coffee bean grinding apparatus further includes: a first inductive coil in electrical communication with the controller; and a second inductive coil in electrical communication with the transmitter; wherein the controller is configured to generate a first electrical current in the first inductive coil which passively induces a second electrical current in the second inductive coil, wherein in response to the second electrical current being induced in the second inductive coil the transmitter transmits the signal.

In certain embodiments, the grinding mechanism includes a first electrical contact in electrical communication with an electrical power source, and the hopper includes a second electrical contact in electrical communication with the transmitter, wherein the transmitter is powered by electrical power received from the electrical power source via the first electrical contact in electrical communication with the second electrical contact.

In certain embodiments, the first and second electrical contacts are spring contacts that are biased into electrical communication together when the hopper is releasably coupled to the grinding mechanism.

In certain embodiments, the hopper includes a cover assembly extending inwardly from an outlet of the hopper, wherein the transmitter is mounted to the cover assembly.

In certain embodiments, the cover assembly includes a shoulder structure which extends from a neck structure, wherein the transmitter is mounted to an underside surface of the shoulder structure.

In certain embodiments, the transmitter is an electromagnetic signal transmitting device configured to transmit an electromagnetic signal, and wherein the receiver is an electromagnetic signal receiving device configured to measure a received signal strength of the electromagnetic signal.

In certain embodiments, the electromagnetic signal transmitting device is a visible light emitting device, the electromagnetic signal is a visible light signal, and the receiver is a photodetector.

In certain embodiments, the visible light emitting device is a light emitting device (LED).

In certain embodiments, the electromagnetic signal transmitting device is an infra-red emitting device, the electromagnetic signal is an infra-red signal, and the receiver is an infra-red sensor.

In certain embodiments, the transmitter is an audio signal transmitting device configured to transmit an audio signal, and wherein the receiver is a transducer device configured to measure a received signal strength of the audio signal.

In certain embodiments, the audio transmitting device is an ultrasonic transmitter, the audio signal is an ultrasonic signal, and the transducer device is an ultrasonic transducer.

In certain embodiments, the controller is configured to: control the receiver to obtain a baseline measurement whilst the transmitter is not transmitting the signal; and determine a difference between the measurement and the baseline measurement, wherein the controller performs the one or more detection functions in response to determining that the difference is equal to or exceeds a difference threshold stored in memory of the controller.

In certain embodiments, the controller is mounted to the grinding mechanism.

In certain embodiments, the hopper includes a carbon dioxide sensor and/or a humidity sensor, in communication with the controller, for measuring a freshness of the coffee beans contained within the hopper, wherein the controller is configured to: compare at least one of a measured level of carbon dioxide and/or a measured humidity level within the hopper against one or more thresholds stored within memory of the controller; and perform one or more freshness detection functions based on the comparison.

In certain embodiments, the coffee bean grinding apparatus further includes a plurality of transmitters and a plurality of receivers, wherein the controller is configured to: receive from each receiver a plurality of measurements indicative of a proportion of a plurality of signals received, the plurality of signals being emitted by the plurality of transmitters; and determine, based on the plurality of measurements, whether the hopper has at least the threshold amount of coffee beans contained therein.

In another aspect, there is provided an espresso machine having an integrated grinding apparatus integrated, wherein the grinding apparatus is configured according to the first aspect and embodiments thereof.

### BRIEF DESCRIPTION OF THE FIGURES

Example embodiments should become apparent from the following description, which is given by way of example only, of at least one preferred but non-limiting embodiment, described in connection with the accompanying figures.

FIG. 1 is a block diagram an example of a controller electrically connected to a plurality of peripheral components of a coffee bean grinding apparatus.

FIG. 2 is a flowchart representing a method performed by a controller of the coffee bean grinding apparatus of FIG. 1.

FIG. 3 is a cross-sectional schematic of an example of a hopper and a portion of a grinding mechanism.

FIG. 4 is a block diagram an example of a controller electrically connected to a plurality of peripheral components of the coffee bean grinding apparatus of FIG. 3.

FIG. 5 is a cross-sectional schematic of an example of a hopper and a portion of a grinding mechanism.

FIG. 6 is a block diagram an example of a controller electrically connected to a plurality of peripheral components of the coffee bean grinding apparatus of FIG. 5.

FIG. 7 is a flowchart representing an example of a method performed by a controller of the coffee bean grinding apparatus of FIGS. 3 and 5.

FIG. 8 is a schematic of an example of a coffee bean grinding apparatus.

FIG. 9 is a perspective view of an espresso coffee making machine including an integrated coffee bean grinding apparatus.

FIG. 10 is a cross-sectional schematic of an example of a hopper and a portion of a grinding mechanism.

### DETAILED DESCRIPTION OF THE EMBODIMENTS

Where reference is made in any one or more of the accompanying drawings to steps and/or features, which have the same reference numerals, those steps and/or features have for the purposes of this description the same function(s) or operation(s), unless the contrary intention appears.

It is to be noted that the discussions contained in the “Background” section and that above relating to prior art arrangements relate to discussions of documents or devices which form public knowledge through their respective publication and/or use. Such should not be interpreted as a representation by the present inventor(s) or the patent applicant that such documents or devices in any way form part of the common general knowledge in the art.

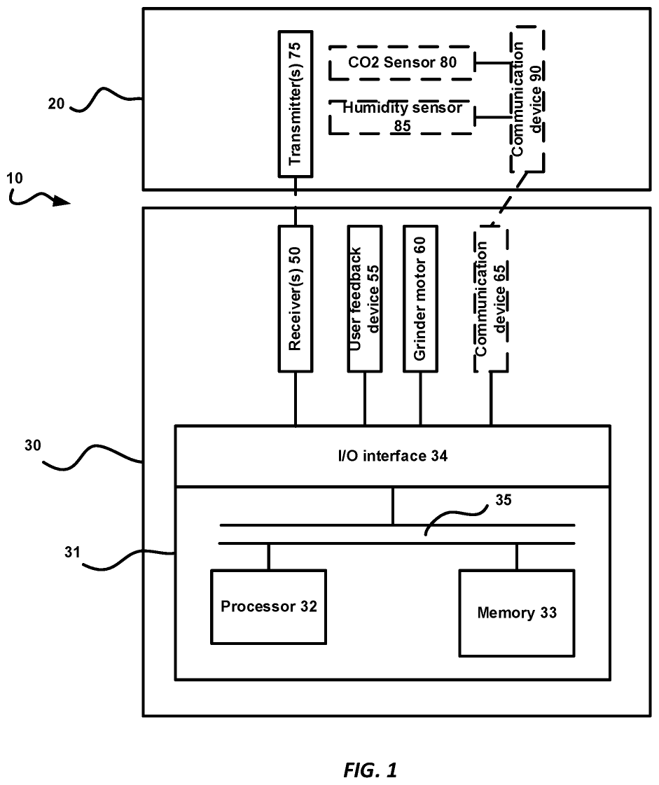

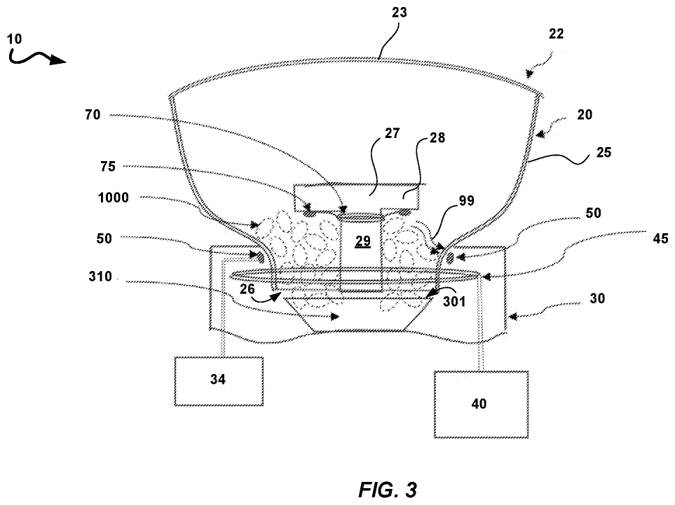

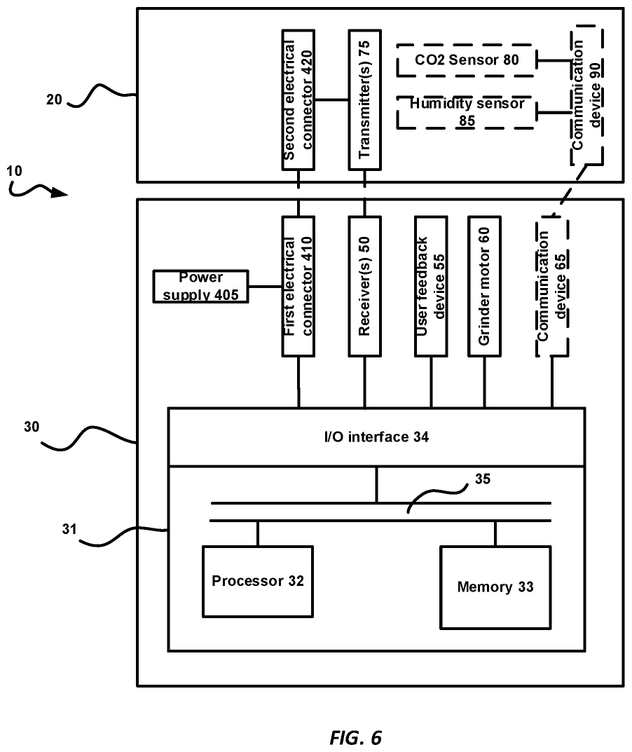

Referring to FIGS. 3 and 6, there is shown a schematic of a coffee bean grinding apparatus 10 for grinding coffee beans 99. The coffee bean grinding apparatus 10 includes a hopper 20 coupled to, preferably releasably coupled to, a grinding mechanism 30. Referring to FIG. 1 there is shown a block diagram of electrical components of the coffee bean grinding apparatus 10 which includes a controller 31 in communication with a receiver 50 and a transmitter 75. At least some of the coffee beans 99 that are contained within the hopper 20 are located between the transmitter 75 and receiver 50. For example, when a sufficient amount of coffee beans 99 are located within the hopper 20, coffee beans 99 are located between a direct/straight path between the transmitter 75 and receiver 50.

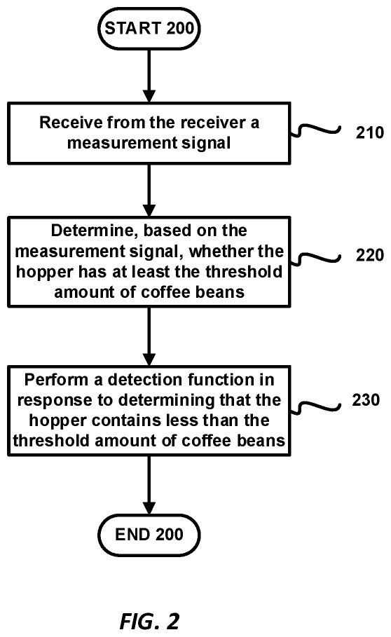

Referring to FIG. 2, there is shown a flowchart representing an example method 200 performed by the controller 31 of the coffee bean grinding apparatus 10. In particular, at step 210 the method 200 includes the controller 31 receiving, from the receiver 50, a measurement indicative of a proportion of the signal 99 emitted by the transmitter 75 which has been received by the receiver. At step 220, the method 200 includes the controller 31 determining, based on the measurement, whether the hopper 20 contains at least a threshold amount of coffee beans. At step 230, in response to determining that the hopper 20 contains less than the threshold amount of coffee beans, the method includes the controller 31 performing one or more detection functions. In some embodiments, the threshold amount of coffee beans may correspond to a one cup or double cup dose, and this threshold amount may be automatically determined or otherwise configurable by the user.

Due to at least some of the coffee beans 1000 contained within the hopper 20 being located between the transmitter 75 and receiver 50, it is possible to detect a proportion of the directly emitted signal 99 rather than indirectly detect a reflection of the emitted signal 99. This configuration results in a higher detection accuracy for determining whether or not a sufficient (or threshold) amount of coffee beans is within the hopper 20.

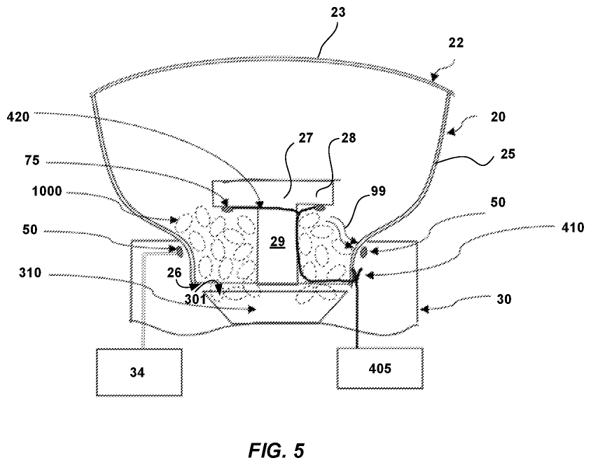

Referring back to FIGS. 3 and 5, the hopper 20 is provided in the form of a container 25 having an outlet 26 for containing the coffee beans 1000. The hopper 20 also includes an inlet 23 provided in the form of an openable lid 22 to fill the container 25 with the coffee beans 1000.

The grinding mechanism 30 includes an inlet 301 in communication with the outlet 26 of the hopper 20 which feeds at least some of the coffee beans 1000 to a burr 310 of the grinding mechanism 30. The burr 310 is driven by a grinder motor 60 which is controlled by the controller 31.

The transmitter 75 is preferably located within the container 25 of the hopper 20. In one form, the transmitter 75 is located in the lower half of the container 25. In one form, the receiver 50 is located within the hopper 20. However, in other embodiments, the receiver 50 is located within a portion of the grinding mechanism 30.

As shown in FIG. 1, the coffee bean grinding apparatus 10 can further include a user feedback device 55 which is in electrical communication with the controller 31. In one form, the controller 31 and the user feedback device 55 can be mounted to the grinding mechanism 30. In another form, the user feedback device 55 may be mounted to the hopper 20. In some embodiments, the user feedback device 55 may be an electronic screen. In additional or alternate embodiments, the user feedback device 55 could be an audio emitting device such as a speaker. In another form, the user feedback device 55 may be one or more lights such as one or more light emitting diodes.

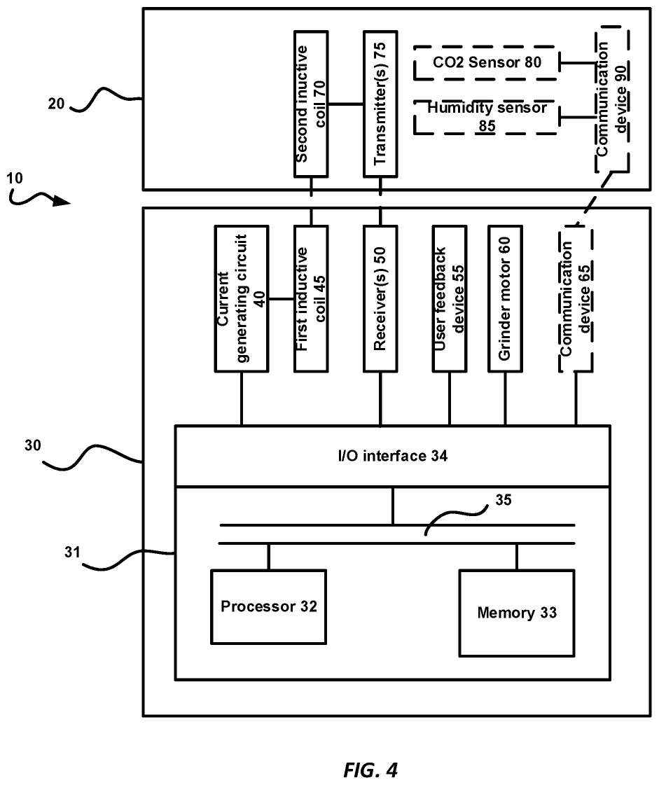

Referring to FIGS. 3 and 4 which shows a more specific implementation of the coffee bean grinding apparatus 10, the coffee bean grinding apparatus 10 can further include a first inductive coil 45 in electrical communication with the controller 31, and a second inductive coil 70 in electrical communication with the transmitter 75. The controller 31 controls a current generating circuit 40 configured to generate a first electrical current in the first inductive coil 45 which passively induces a second electrical current in the second inductive coil 70. In response to the second electrical current being induced in the second inductive coil 70, the transmitter 75 transmits the signal 99. In this implementation, the first and second inductive coils 45, 70 are positioned in close proximity to one another to achieve the inductive coupling between the coils when the hopper 20 and the grinding mechanism 30 are releasably coupled together.

Referring to FIGS. 5 and 6, there is shown an alternate example implementation of the coffee bean grinding apparatus 10. In particular, the grinding mechanism 20 includes a first electrical connector 410 in electrical communication with an electrical power supply 405, and the hopper 20 includes a second electrical contact 420 in electrical communication with the transmitter 75. The transmitter 75 is electrically powered by electrical power received from the electrical power supply 405 via the first and second electrical connectors 410, 420 which are in electrical communication with each other. In one form, the electrical power supply 405 is in electrical communication with the controller 31, wherein the first electrical connector 410 is in electrical communication with the controller 31. The first and second electrical connectors 410, 420 can be spring contacts that are biased into electrical communication with each other when the hopper 20 is releasably coupled to the grinding mechanism 30.

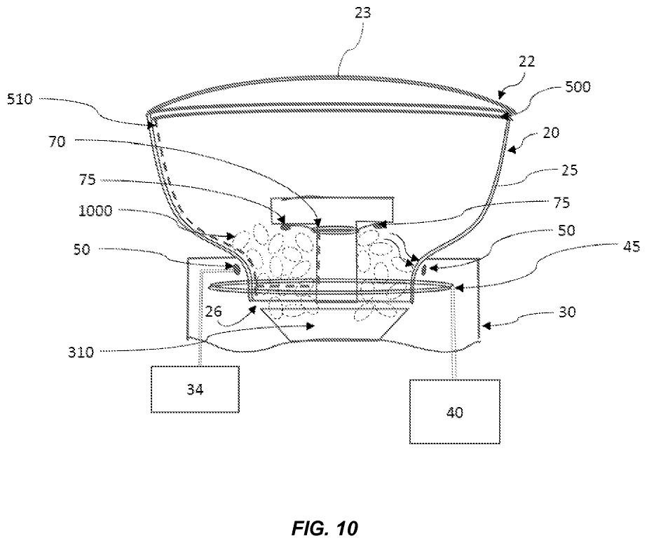

Referring to FIG. 10, there is shown another alternate example implementation of the coffee bean grinding apparatus 10. In this example, the coffee bean grinding apparatus 10 functions in a similar manner to the example as shown in FIGS. 3 and 4, with like reference numerals used to indicate like features. In this example, the user feedback device 55 of the coffee bean grinding apparatus 10 is provided by a light ring 500 mounted on the hopper 20, with the light ring 500 being in electrical communication with the first inductive coil 45 by way of a wire or electrical connector 510. The light ring 500 may be in the form of a ring of light emitting diode (LED), for example. In embodiments, the light ring 500 may be mounted or otherwise disposed at any location in, on, or around the hopper 20 that may add to the aesthetics of the coffee bean grinding apparatus 10, to improve the visibility of the contents of the hopper 20 (e.g. amount of coffee beans), or to otherwise accompany the one or more detection functions performed by the controller 31. It will also be appreciated that in a preferred form, and as depicted in FIG. 10, the light ring 500 is provided in the form or structure of a ring that substantially encircles an inner or outer wall of the hopper 20. However, the form or the structure of the light ring 500 is not necessarily limited to this configuration, and the light ring 500 may alternatively be provided in the form of a light bar, a light panel, or any other physical configuration or shape to suit the design requirements of the coffee bean grinding apparatus 10.

As earlier described, the first inductive coil 45 is in turn in electrical communication with the controller 31. The controller 31 controls a current generating circuit 40 configured to generate a first electrical current in the first inductive coil 45 which passively induces a second electrical current in the second inductive coil 70. In response to the second electrical current being induced in the second inductive coil 70, the transmitter 75 transmits the signal 99. In this implementation, the first and second inductive coils 45, 70 are positioned in close proximity to one another to achieve the inductive coupling between the coils when the hopper 20 and the grinding mechanism 30 are releasably coupled together.

In the example of the coffee bean grinding apparatus 10 of FIG. 10, it is envisaged that the controller 31 may control the operation of the light ring 500 in response to the measurement indicative of a proportion of the signal emitted by the transmitter 75 which has been received by the receiver 50. That is, the operation of the light ring 500 may be controlled by the controller 31 in response to a determination, based on the measurement, of the amount of coffee beans in the hopper 20. In one example, the light ring 500 may be operated to blink or flash in response to a determination, based on the measurement, that the amount of coffee beans in the hopper 20 is below the threshold amount, thereby indicating to the user that the hopper 20 needs to be refilled. The operation of the light ring 500 (which may include the blink or flash rate) may be controlled by an electronic circuit associated with the hopper 20. Alternatively, the blinking or flashing of the light ring 500 may be achieved by the controller 31 controlling the power (i.e. switching on and off) to the first and/or second inductive coils 45, 70. It will be appreciated that the operation of the light ring 500 is not necessarily limited to blinking or flashing functions, and may, in one example, be controlled to steadily emit a particular light colour to indicate an associated amount of coffee beans in the hopper 20. By way of non-limiting example, the light ring 500 may be controlled to emit a red light in response to a determination, based on the measurement, that the amount of coffee beans in the hopper 20 is below the threshold amount, and to emit a white light in response to a determination, based on the measurement, that the amount of coffee beans in the hopper 20 is above the threshold amount.

As shown in the examples of the coffee bean grinding apparatus 10 of FIGS. 3, 5, and 10, the hopper 20 can include a cover assembly 27 extending upwardly and inwardly from the outlet 26 within the container 25, wherein the transmitter 75 is mounted to the cover assembly 27. The cover assembly 27 includes a shoulder structure 29 which extends from a neck structure 28, wherein the transmitter 75 is mounted to an underside surface of the shoulder structure 29. In other embodiments (not shown), the transmitter 75 may be mounted to the neck structure 28. The shoulder structure 29 helps to disperse the coffee beans 1000 within the container 25 as well as protect a user from injury by inhibiting access to the burr 310 of the grinding mechanism 30 when in use. It will be appreciated that the shoulder structure 29 may also hide the various electrical components (transmitter, coils, electrical connectors) from view, and to also inhibit user access to such components.

The transmitter 75 of the examples of the coffee bean grinding apparatus 10 of FIGS. 3, 5, and 10 is an electromagnetic signal transmitting device configured to transmit an electromagnetic signal 99. The receiver 50 is an electromagnetic signal receiving device configured to measure a received signal strength of the electromagnetic signal 99 emitted by the transmitter 75. In one form, the electromagnetic signal transmitting device is a visible light emitting device, the electromagnetic signal 99 is a visible light signal, and the receiver 50 is a photodetector. For example, the visible light emitting device can be provided in the form of a light emitting device (LED). In an alternate embodiment, the electromagnetic signal transmitting device is an infra-red emitting device, the electromagnetic signal 99 is an infra-red signal, and the receiver 50 is an infra-red sensor. In another alternate embodiment, the transmitter 75 is an audio transmitting device configured to transmit an audio signal 99, wherein the receiver 50 is a transducer device configured to measure a received signal strength of the audio signal 99. In an alternate configuration to the electromagnetic signal implementation, the audio transmitting device is an ultrasonic transmitter, the audio signal 99 is an ultrasonic signal, and the transducer device is an ultrasonic transducer. In one form, the transmitter 75 could be a beacon transmitter which periodically transmits the signal 99 and thus does not need to be controlled by the controller 31 to generate the signal 99.

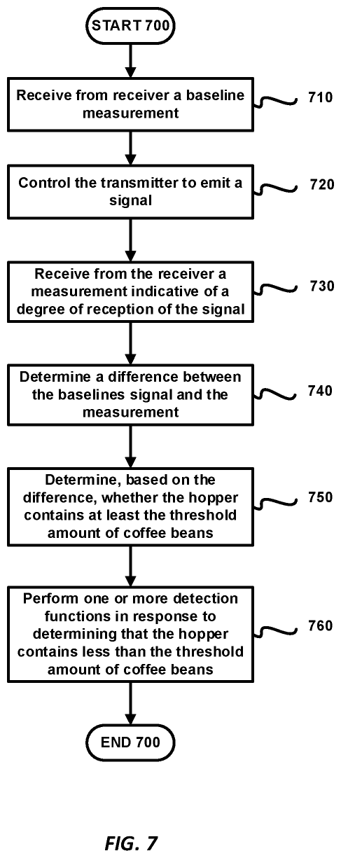

Referring to FIG. 7, there is shown a flowchart representing a more specific example of a method 700 performed by the controller 31 of the coffee bean grinding apparatus 10. In particular, at step 710 the method 700 includes the controller 31 controlling the receiver to obtain a baseline measurement whilst the transmitter 75 is not transmitting the signal 99. At step 720, the method 700 includes the controller 31 controlling the transmitter 75 to transmit the signal 99. At step 730, the method 700 includes the controller 31 receiving a measurement signal indicative of a proportion of the signal received by the receiver. At step 740, the method 700 includes the controller 31 determining a difference between the measurement and the baseline measurement. At step 750, the method 700 includes the controller 31 determining whether the difference is equal to or exceeds a difference threshold stored in memory 33 of the controller 31. At step 760, the method 700 includes the controller 31 performing one or more detection functions in response to determining that the hopper does not contain the threshold amount of coffee beans. Advantageously, a new baseline measurement is obtained each time the method 700 is performed. Thus, coffee bean powder which may be contained within the hopper 20 will have a reduced effect on the accuracy of the sensing process. In one form, the method 700 may be performed each time a grinding action is to be performed by the coffee bean grinding apparatus 10. For example, the coffee bean grinding apparatus 10 can include an input device such as a touch screen display or button allowing the user to provide input to request the coffee bean grinding apparatus 10 to grind at least some of the coffee beans 1000. In response, the receiving the request via the input device, the controller 31 performs method 700.

As depicted in FIGS. 4 and 6 by broken line, the hopper 20 can optionally include a carbon dioxide sensor 80 and/or a humidity sensor 85, in electrical communication with a communication device 90 which in turn is in communication with a communication device 65 of the controller 31. The communication devices 65, 90 may be a physical or wireless communication interface. Measurements received by the controller 31 from the carbon dioxide sensor 80 and/or a humidity sensor 85 can be used for measuring a freshness of the coffee beans contained within the hopper. In one embodiment, the controller 31 can be configured to compare at least one of a measured level of carbon dioxide and/or a measured humidity level within the hopper against one or more thresholds stored within memory 33 of the controller 31. The controller 31 can be configured to perform one or more freshness detection functions based on the comparison. In one form, the one or more freshness detection functions could include presenting a user alert via the user feedback device, emitting an audio signal via the user feedback device, and/or flashing one or more lights (for example, as described above in relation to the embodiment of FIG. 10).

Whilst the embodiments described above have been in relation to a single transmitter and receiver pair, it is possible, as shown in FIGS. 3, 5, and 10, that the coffee bean grinding apparatus includes plurality of transmitter/receiver pairs including a plurality of transmitters and a plurality of receivers. In particular, the hopper includes a plurality of transmitters and the grinding mechanism includes a plurality of receivers. The controller is configured to control the plurality of transmitters to emit a plurality of signals 99, receive from each receiver a plurality of measurements indicative of a degree of reception of each signal 99, and determine, based on the plurality of measurements, whether the hopper has at least the threshold amount of coffee beans contained in the hopper. In one form, the controller may average the measurements detected prior to determining the difference. Alternatively, if a single transmitter/receiver pair result in a difference measurement that is indicative of an insufficient amount of coffee beans in the container, the user alert is provided via the user feedback device despite one or more other transmitter/receiver pairs having an acceptable difference.

The one or more detection (or alert) functions include the controller controlling the user feedback device to provide an alert indicative of determining that the hopper contains less than the threshold amount of coffee beans. In embodiments where the user feedback device 55 is an electronic screen, the controller can control the user feedback device 55 to present a user alert indicative of the detection. In an additional or alternate embodiments where the user feedback device is a transducer, the controller can control the user feedback device to emit an audio signal to indicate to the user that the hopper needs to be filled with more coffee beans. In an additional or alternate form, and as described above in relation to FIG. 10, the user feedback device may be a plurality of lights, such as LEDS, which are controlled by the controller 31 to emit light in a manner (e.g. flash) to indicate the user alert.

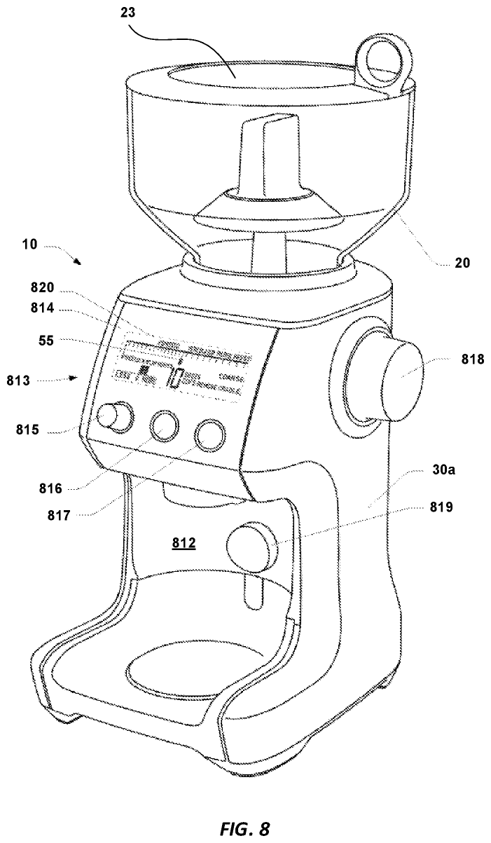

Referring to FIG. 8, there is shown a perspective view of an example of the coffee bean grinding apparatus 10. The coffee bean grinding apparatus 10 is an electrical, motorised coffee grinder which comprises a base 30a housing the grinding mechanism 30. The base is releasably coupled to the hopper 20. The base 32 has a recess or discharge area 812 into which ground coffee is dispensed. The discharge area 812 can accommodate containers such as a portafilter, filter or storage canister. The base 30 has a head 813 located above the recess 812. A front panel or surface 814 of the head 813 features various user controls including (as will be further explained) a discharge amount adjustment rotating knob 815, a push button or one or more other user controls for choosing discreet preset discharge amounts 816, a start/cancel button 817 and a grind size selector dial 818. The grind size selector dial mechanically controls the vertical movement of the upper burr of the grinder 10. The spacing between the upper burr and the lower burr determines the grind size. The dial 818 also controls the appearance of the display 820 by causing one of a number of arrow icons to appear in the appropriate position under a grind size index line. The preset amount button 816 allows the user to choose an amount of coffee grinds to discharge. Depressing this button causes a numeric display portion to change in discrete increments. Each displayed numeric value represents a grinding time for each grind type. Parameters such as grinding time and grinding type are related to the discharge amount in accordance with a look up table stored in the memory 33. A rear surface of the recess 812 also has an external button 819 coupled to an electrical switch that is activated with the presence of a portafilter. The front panel 814 also features an electronic display 820. Further details in relation to the coffee bean grinding apparatus are disclosed in PCT/AU2011/000274, the contents of which is herein incorporated by reference in its entirety.

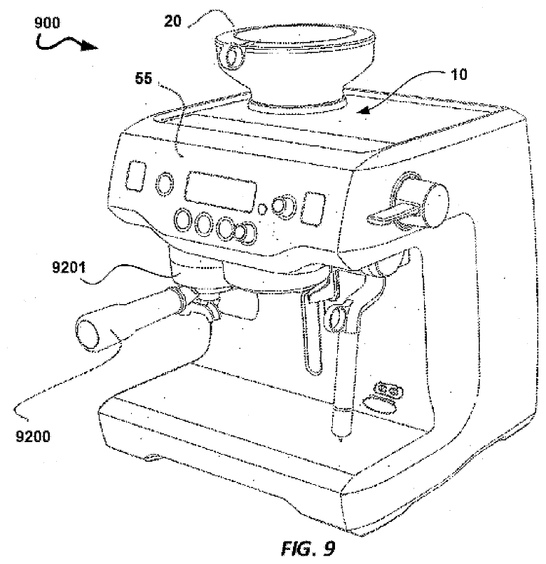

Referring to FIG. 9, there is shown a schematic of an espresso making machine 900 having an integrated coffee bean grinding apparatus 10. The integrated coffee bean grinding apparatus 10 of the espresso coffee making machine 900 can be configured according to any of the above described example embodiments. Referring more specifically to FIG. 9, the espresso coffee making machine 900 may incorporate a tamping augur for filling a portafilter 9200 engaged with a fill head 9201. The fill head 9201 receives ground coffee from the grinder 10 and discharges it into the portafilter 9200. Further details regarding the espresso coffee making machine are disclosed by PCT/AU2014/000378, the contents of which is herein incorporated by reference in its entirety.

Although the invention has been described with reference to a preferred embodiment, it will be appreciated by those skilled in the art that the invention may be embodied in other forms.

The advantageous embodiments and/or further developments of the above disclosure—except for example in cases of clear dependencies or inconsistent alternatives—can be applied individually or also in arbitrary combinations with one another.

1. A coffee bean grinding apparatus including:
a transmitter for emitting a signal;
a receiver for receiving at least a portion of the signal;
a hopper for containing coffee beans, wherein at least a portion of the coffee beans contained by the hopper is located between the transmitter and receiver;
a grinding mechanism for grinding the coffee beans fed by the hopper; and
a controller, in communication with the transmitter, the receiver and the grinding mechanism, configured to:
receive, from the receiver, a measurement indicative of a proportion of the signal received by the receiver;
determine, based on the measurement, whether the hopper contains at least a threshold amount of coffee beans; and
in response to determining that the hopper contains less than the threshold amount of coffee beans, perform one or more detection functions.

2. The coffee bean grinding apparatus according to claim 1, wherein the transmitter is located within the hopper.

3. The coffee bean grinding apparatus according to claim 1, wherein the receiver is located:
within the hopper; or
within the grinding mechanism coupled to the hopper.

4. The coffee bean grinding apparatus according to claim 1, wherein the coffee bean grinding apparatus includes a user feedback device, wherein the one or more detection functions include the controller controlling the user feedback device to provide an alert indicative of determining that the hopper contains less than the threshold amount of coffee beans.

5. The coffee bean grinding apparatus according to claim 1, wherein the coffee bean grinding apparatus further includes:
a first inductive coil in electrical communication with the controller; and
a second inductive coil in electrical communication with the transmitter;
wherein the controller is configured to generate a first electrical current in the first inductive coil which passively induces a second electrical current in the second inductive coil, wherein in response to the second electrical current being induced in the second inductive coil the transmitter transmits the signal.

6. The coffee bean grinding apparatus according to claim 1, wherein the grinding mechanism includes a first electrical contact in electrical communication with an electrical power source, and the hopper includes a second electrical contact in electrical communication with the transmitter, wherein the transmitter is powered by electrical power received from the electrical power source via the first electrical contact in electrical communication with the second electrical contact.

7. The coffee bean grinding apparatus according to claim 6, wherein the first and second electrical contacts are spring contacts that are biased into electrical communication together when the hopper is releasably coupled to the grinding mechanism.

8. The coffee bean grinding apparatus according to claim 1, wherein the hopper includes a cover assembly extending inwardly from an outlet of the hopper, wherein the transmitter is mounted to the cover assembly.

9. The coffee bean grinding apparatus according to claim 8, wherein the cover assembly includes a shoulder structure which extends from a neck structure, wherein the transmitter is mounted to an underside surface of the shoulder structure.

10. The coffee bean grinding apparatus according to claim 1, wherein the transmitter is an electromagnetic signal transmitting device configured to transmit an electromagnetic signal, and wherein the receiver is an electromagnetic signal receiving device configured to measure a received signal strength of the electromagnetic signal.

11. The coffee bean grinding apparatus according to claim 10, wherein the electromagnetic signal transmitting device is a visible light emitting device, the electromagnetic signal is a visible light signal, and the receiver is a photodetector.

12. The coffee bean grinding apparatus according to claim 11, wherein the visible light emitting device is a light emitting device (LED).

13. The coffee bean grinding apparatus according to claim 10, wherein the electromagnetic signal transmitting device is an infra-red emitting device, the electromagnetic signal is an infra-red signal, and the receiver is an infra-red sensor.

14. The coffee bean grinding apparatus according to claim 1, wherein the transmitter is an audio signal transmitting device configured to transmit an audio signal, and wherein the receiver is a transducer device configured to measure a received signal strength of the audio signal.

15. The coffee bean grinding apparatus according to claim 14, wherein the audio transmitting device is an ultrasonic transmitter, the audio signal is an ultrasonic signal, and the transducer device is an ultrasonic transducer.

16. The coffee bean grinding apparatus according to claim 1, wherein the controller is configured to:
control the receiver to obtain a baseline measurement whilst the transmitter is not transmitting the signal; and
determine a difference between the measurement and the baseline measurement, wherein the controller performs the one or more detection functions in response to determining that the difference is equal to or exceeds a difference threshold stored in memory of the controller.

17. The coffee bean grinding apparatus according to claim 1, wherein the controller is mounted to the grinding mechanism.

18. The coffee grinding apparatus according to claim 1, wherein the hopper includes a carbon dioxide sensor and/or a humidity sensor, in electrical communication with the controller, for measuring a freshness of the coffee beans contained within the hopper, wherein the controller is configured to:
compare at least one of a measured level of carbon dioxide and/or a measured humidity level within the hopper against one or more thresholds stored within memory of the controller; and
perform one or more freshness detection functions based on the comparison.

19. The coffee grinding apparatus according to claim 1, further including a plurality of transmitters and a plurality of receivers, wherein the controller is configured to:
receive from each receiver a plurality of measurements indicative of a proportion of a plurality of signals received, the plurality of signals being emitted by the plurality of transmitters; and
determine, based on the plurality of measurements, whether the hopper has at least the threshold amount of coffee beans contained therein.

20. An espresso machine having an integrated grinding apparatus integrated, wherein the grinding apparatus is configured according to claim 1.
